(19) United States
(12) Patent Application Publication
(10) Pub. No.: US 2009/0143416 A1
(43) Pub. Date: Jun. 4, 2009

(54) Method of Inducing Tolerance of Plants Against Bacterioses

(76) Inventors: Harald Kohle, Bobenheim (DE); Ted R. Bardinelli, Durham, NC (US); Marco-Antonio Tavares-Rodrigues, Sao Paulo (BR)

Correspondence Address: BIRCH STEWART KOLASCH & BIRCH, PO BOX 747, FALLS CHURCH, VA, 22040-0747, US

(21) Appl. No.: 12/223,029

(22) PCT Filed: Mar. 6, 2007
(86) PCT No.: PCT/EP2007/052101

§ 371 Date: Jul. 21, 2008

Related U.S. Application Data
(60) Provisional application No. 60/782,184, filed on Mar. 14, 2006.

(51) Int. Cl. A61K 31/4152; A61K 31/4196; A61P 37/04; A61K 31/505

(52) U.S. Cl. 514/269; 514/384; 514/407

(57) ABSTRACT

A method of inducing tolerance of plants against bacterioses which comprises treating the plants, the soil or seeds with an effective amount of a combination of a compound of the formula I

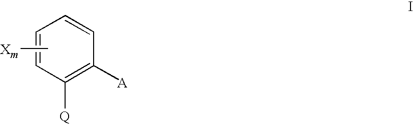

in which
X is halogen, alkyl or trifluoromethyl;
m is 0 or 1;
Q is C(═CH—CH₃)—COOCH₃, C(═CH—OCH₃)—COOCH₃, C(═N—OCH₃)—CONHCH₃, C(═N—OCH₃)—COOCH₃, N(—OCH₃)—COOCH₃, or a group Q1

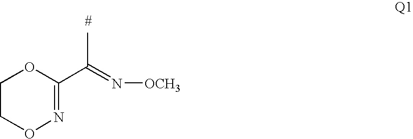

wherein # denotes the bond to the phenyl ring;
A is —O—B, —CH₂O—B, —OCH₂—B, —CH₂S—B, —CH═CH—B, —C≡C—B, —CH₂O—N═C(R¹)—B, —CH₂S—N═C(R¹)—B, —CH₂O—N═C(R¹)—CH═CH—B, or —CH₂O—N═C(R¹)—C(R²)═N—OR³, where
   B is phenyl, naphthyl, 5- or 6-membered hetaryl or 5- or 6-membered heterocyclyl, containing one to three N atoms and/or one O or S atom or one or two O and/or S atoms, the ring systems being unsubstituted or substituted as defined in the description;
   R¹ is hydrogen, cyano, alkyl, haloalkyl, cycloalkyl, alkoxy, or alkylthio;
   R² is phenyl, phenylcarbonyl, phenylsulfonyl, 5- or 6-membered hetaryl, 5- or 6-membered hetarylcarbonyl or 5- or 6-membered hetarylsulfonyl, the ring systems being unsubstituted or substituted by one to three radicals R^{a}, C₁-C₁₀-alkyl, C₃-C₆-cycloalkyl, C₂-C₁₀-alkenyl, C₂-C₁₀-alkynyl, C₁-C₁₀-alkyl-carbonyl, C₂-C₁₀-alkenylcarbonyl, C₃-C₁₀-alkynylcarbonyl, C₁-C₁₀-alkyl-sulfonyl, or C(═NOR^{A})—R^{B}, the hydrocarbon radicals of these groups being unsubstituted or substituted as defined in the description;
   R³ is hydrogen, C₁-C₆-alkyl, C₂-C₆-alkenyl, C₂-C₆-alkynyl, the hydrocarbon radicals of these groups being unsubstituted or substituted as defined in the description;
      and a second active compound as defined in the description; which is taken up by the plants or seeds.

The present invention relates to a method of inducing tolerance of plants against bacterioses, which comprises treating the plants, the soil or the seeds with an effective amount of a compound of the formula I,

1) a strobilurin compound of the formula I

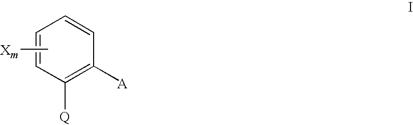

in which
X is halogen, C₁-C₄-alkyl or trifluoromethyl;
m is 0 or 1;
Q is C(═CH—CH₃)—COOCH₃, C(═CH—OCH₃)—COOCH₃, C(═N—OCH₃)—CONHCH₃, C(═N—OCH₃)—COOCH₃, N(—OCH₃)—COOCH₃, or a group Q1

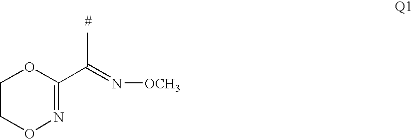

wherein # denotes the bond to the phenyl ring;
A is —O—B, —CH₂O—B, —OCH₂—B, —CH═CH—B, —C≡C—B, —CH₂O—N═C(R¹)—B, —CH₂O—N═C(R¹)—CH═CH—B, or —CH₂O—N═C(R¹)—C(R²)═N—OR³, where
B is phenyl, naphthyl, 5- or 6-membered hetaryl or 5- or 6-membered heterocyclyl, containing one to three N atoms and/or one O or S atom or one or two O and/or S atoms, the ring systems being unsubstituted or substituted by one to three radicals R^{a}:
   R^{a} is cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylamino-carbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered hetaryl, 5- or 6-membered hetaryloxy, C(═NOR^{a})—R^{b} or OC(R^{a})₂—C(R^{b})═NOR^{b}, the cyclic radicals, in turn, being unsubstituted or substituted by one to three radicals R^{b}:
      R^{b} is cyano, nitro, halogen, amino, aminocarbonyl, aminothio-carbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-halo-alkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkyl-amino-carbonyl, C₁-C₆-alkylaminothiocarbonyl, di-C₁-C₆-alkyl-aminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered heterocyclyl, 5- or 6-membered hetaryl, 5- or 6-membered hetaryloxy or C(═NOR^{A})—R^{B};
R^{A}, R^{B} are hydrogen or C₁-C₆-alkyl;
R¹ is hydrogen, cyano, C₁-C₄-alkyl, C₁-C₄-haloalkyl, C₃-C₆-cycloalkyl, or C₁-C₄-alkoxy;
R² is phenyl, phenylcarbonyl, phenylsulfonyl, 5- or 6-membered hetaryl, 5- or 6-membered hetarylcarbonyl or 5- or 6-membered hetarylsulfonyl, the ring systems being unsubstituted or substituted by one to three radicals R^{a},
   C₁-C₁₀-alkyl, C₃-C₆-cycloalkyl, C₂-C₁₀-alkenyl, C₂-C₁₀-alkynyl, C₁-C₁₀-alkyl-carbonyl, C₂-C₁₀-alkenylcarbonyl, C₃-C₁₀-alkynylcarbonyl, C₁-C₁₀-alkyl-sulfonyl, or C(═NOR^{A})—R^{B}, the hydrocarbon radicals of these groups being unsubstituted or substituted by one to three radicals R^{c}:
   R^{c} is cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkyl-thio, C₁-C₆-alkylamino, di-C₁-C₆-alkylamino, C₁-C₆-alkylamino-carbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy,
      C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, 5- or 6-membered hetaryloxy and hetarylthio, it being possible for the cyclic groups, in turn, to be partially or fully halogenated or to have attached to them one to three radicals R^{a}; and
R³ is hydrogen, C₁-C₆-alkyl, C₂-C₆-alkenyl, C₂-C₆-alkynyl, the hydrocarbon radicals of these groups being unsubstituted or substituted by one to three radicals R^{c};
   and
   2) a compound selected from the groups A) to D):
A) antibiotics: cycloheximid, griseofulvin, kasugamycin, natamycin, polyoxin and streptomycin,
B) bactericides: bronopol, cresol, dichlorophen, dipyrithione, dodicin, fenaminosulf, formaldehyde, hydrargaphen, 8-hydroxyquinoline sulfate, nitrapyrin, octhilinone, oxolinic acid, oxytetracycline, probenazole, tecloftalam, thiomersal;
C) famoxadone and cymoxanil, and
D) copper, silver, and zinc salts, such as Bordeaux mixture, copper acetate, copper oxychloride, basic copper sulfate;

In a synergistically effective amount,

which components 1) and 2) are taken up by the plants or seeds. In addition, the invention generally relates to the use of the combinations of a compound of formula I and a compound selected from the group A), B), C), and D) for immunizing plants against bacterioses.

Bacteria are predominantly found in moderate and humid-warm climatic regions as pathogens of diseases (bacterioses) in a large number of crop plants. Occasionally, these diseases cause substantial economic damage. Examples which are generally known are the death of entire fruit plantations caused by a variety of Erwinia species (“fireblight” in pears and apples), and bacterial soft rot in potatoes and many other plants, various plant tumors triggered by agrobacteria, and the necroses on a variety of vegetables, on rice, wheat and citrus fruit, caused by Xanthomonas species. The bacterioses caused by Pseudomonas species, in particular in vegetables, top fruit species and tobacco are especially dreaded.

As can be expected, conventional fungicides, which interfere with fungus-specific metabolic processes, are not active against bacterioses. Thus, the only way of controlling them which has been possible to date was the use of antibiotics, but this procedure is rarely practiced: the extensive use of antibiotics in agriculture is debated since, in principle, these antibiotics rely on the same mechanisms of action as are used against bacterial pathogens in human and veterinary medicine. They may thus favor the buildup of resistances. Moreover, antibiotics are expensive, owing to their molecular structures (most of which are complicated) and can only be produced by biotechnological methods. Therefore, it is an object of the invention to reduce the necessity to use antibiotics in agriculture.

An alternative way to prevent bacterial diseases in plants is taught in WO 03/075663: Strobilurine type fungicides have a stimulatory effect on the plants' intrinsic immune system against bacteria. However, this effect is not always fully satisfactory.

It is known that the famoxadone-cymoxanil combination product Tanos™ of DuPont can be used not only for controlling fungal diseases but also for a disease suppression of certain bacterial crop diseases.

Furthermore it is known that salts of certain metal cations have a bactericidal activity. In crop protection the use of copper salts is well established for many years. The anions of such salts are not critical for the bactericidal activity, for practical reasons hydroxids, oxids, or anions of mineral acids or organic acids are used, such as carbonic acids or sulfonic acids.

It is an object of the present invention to provide a highly effective method, which can be used broadly, which does not damage the plants and which brings about increased tolerance of the plants against phytobacterioses at a reduced total amount of active compounds applied.

Although it is generally known that the active compounds mentioned as component 2) above have an activity against bacteria, and can be used for controlling such diseases, there is not any teaching that the component 2) compounds might increase the strobilurins' stimulatory effect on the plants' intrinsic immune system against bacteria.

We have found that this object is achieved by the method defined at the outset.

The above-mentioned strobilurines of formula I are known as fungicides and, in some cases, also as insecticides (EP-A 178 826; EP-A 253 213; WO 93/15046; WO 95/18789; WO 95/21153; WO 95/21154; WO 95/24396; WO 96/01256; WO 97/15552).

The active compounds according to the groups A) to D) mentioned above, their preparation and their action against harmful fungi, and bacteria, resp., are generally known in the art (cf.: http://www.hclrss.demon.co.uk/index.html; The Pesticide Manual, 10th Ed., BCPC, 1995); the compounds with common name are commercially available.

The good compatibility, with plants, of the active ingredients of the formula I at the concentrations required for controlling plant diseases permits the treatment of aerial plant parts and also the treatment of propagation material and seed, and of the soil.

In the method according to the invention, the active ingredients are taken up by the plant either through the leaf surface or through the roots and is distributed within the entire plant in the sap.

Thus, the protective action after carrying out the method according to the invention is not just found in those plant parts, which have been sprayed directly, but the tolerance to bacterial diseases of the entire plant is increased.

In a preferred embodiment of the method, the aerial plant parts are treated with a formulation or with a tank mix of the active ingredients 1) and 2).

Especially preferred for the method according to the invention are active ingredients with the following meanings of the substituents, in each case alone or in combination, the disclosure of the publications cited being hereby incorporated:

Especially preferred for the method according to the invention are, as component 1, the active ingredients of the formulae II to VIII, in which

### V is OCH₃ and NHCH₃,

### Y is CH and N, and

T and Z independently of one another are CH and N.

Preferred active ingredients of the formula I in which Q is N(—OCH₃)—COOCH₃ are the compounds described in the publications WO 93/15046 and WO 96/01256.

Preferred active ingredients of the formula I in which Q is C(═CH—OCH₃)—COOCH₃ are the compounds described in the publications EP-A 178 826 and EP-A 278 595.

Preferred active ingredients of the formula I in which Q is C(═N—OCH₃)—COOCH₃ are the compounds described in the publications EP-A 253 213 and EP-A 254 426.

Preferred active ingredients of the formula I in which Q is C(═N—OCH₃)—CONHCH₃ are the compounds described in the publications EP-A 398 692, EP-A 477 631 and EP-A 628 540.

Preferred active ingredients of the formula I in which Q is C(═CH—CH₃)—COOCH₃ are the compounds described in the publications EP-A 280 185 and EP-A 350 691.

Preferred active ingredients of the formula I in which Q is —CH₂O—N═C(R¹)—B are the compounds described in the publications EP-A 460 575 and EP-A 463 488.

Preferred active ingredients of the formula I in which A is —O—B are the compounds described in the publications EP-A 382 375 and EP-A 398 692.

Preferred active ingredients of the formula I in which A is —CH₂O—N═C(R¹)—C(R²)═N—OR³ are the compounds described in the publications WO 95/18789, WO 95/21153, WO 95/21154, WO 97/05103 and WO 97/06133.

Especially preferred are the active ingredients of the formula I in which

### Q is N(—OCH₃)—COOCH₃,

### A is CH₂—O— and

B is 3-pyrazolyl or 1,2,4-triazolyl, where B has attached to it one or two substituents selected from the group of
halogen, methyl and trifluoromethyl and
phenyl and pyridyl, in particular 2-pyridyl, substituted by 1 to 3 radicals R^{b}.

These active ingredients are described by formula II,

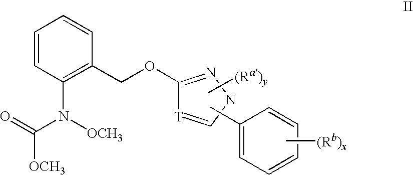

in which T is a carbon or a nitrogen atom, R^{a′} is halogen, methyl and trifluoromethyl, y is zero, 1 or 2, R^{b} is as defined for formula I, x is zero, 1, 2, 3 or 4.

More preferred active ingredients are those of formula II′:

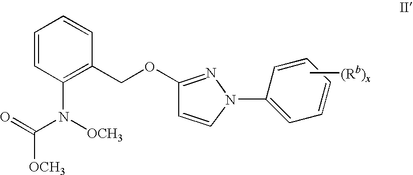

in which R^{b} is as defined for formula I.

With regard to their use, the compounds compiled in the tables, which follow, are especially preferred.

TABLE I

| | |
|---|---|
| | II |
| 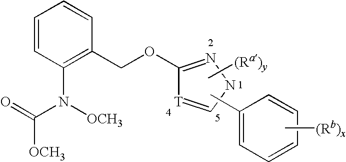 | |

| | | | Position of | | |
|---|---|---|---|---|---|
| | | | the group | | |
| No. | T | (R^{a′})_{y} | phenyl-(R^{b})ₓ | (R^{b})ₓ | Reference |
| I-1 | N | — | 1 | 2,4-Cl₂ | WO 96/01256 |
| I-2 | N | — | 1 | 4-Cl | WO 96/01256 |
| I-3 | CH | — | 1 | 2-Cl | WO 96/01256 |
| I-4 | CH | — | 1 | 3-Cl | WO 96/01256 |
| I-5 | CH | — | 1 | 4-Cl | WO 96/01256 |
| I-6 | CH | — | 1 | 4-CH₃ | WO 96/01256 |
| I-7 | CH | — | 1 | H | WO 96/01256 |
| I-8 | CH | — | 1 | 3-CH₃ | WO 96/01256 |
| I-9 | CH | 5-CH₃ | 1 | 3-CF₃ | WO 96/01256 |
| I-10 | CH | 1-CH₃ | 5 | 3-CF₃ | WO 99/33812 |
| I-11 | CH | 1-CH₃ | 5 | 4-Cl | WO 99/33812 |
| I-12 | CH | 1-CH₃ | 5 | — | WO 99/33812 |

TABLE II

| | |
|---|---|
| | III |
| 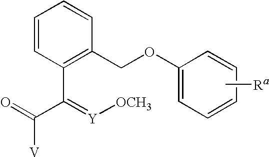 | |

| No. | V | Y | R^{a} | Reference |
|---|---|---|---|---|
| II-1 | OCH₃ | N | 2-CH₃ | EP-A 253 213 |
| II-2 | OCH₃ | N | 2,5-(CH₃)₂ | EP-A 253 213 |
| II-3 | NHCH₃ | N | 2,5-(CH₃)₂ | EP-A 477 631 |
| II-4 | NHCH₃ | N | 2-Cl | EP-A 398 692 |
| II-5 | NHCH₃ | N | 2-CH₃ | EP-A 398 692 |
| II-6 | NHCH₃ | N | 2-CH₃, | EP-A 628 540 |
| | | | 4-OCF₃ | |
| II-7 | NHCH₃ | N | 2-Cl, | EP-A 628 540 |
| | | | 4-OCF₃ | |
| II-8 | NHCH₃ | N | 2-CH₃, | EP-A 11 18 609 |
| | | | 4-OCH(CH₃)—C(CH₃)═NOCH₃ | |
| II-9 | NHCH₃ | N | 2-Cl, | EP-A 11 18 609 |
| | | | 4-OCH(CH₃)—C(CH₃)═NOCH₃ | |
| II-10 | NHCH₃ | N | 2-CH₃, | EP-A 11 18 609 |
| | | | 4-OCH(CH₃)—C(CH₂CH₃)═NOCH₃ | |
| II-11 | NHCH₃ | N | 2-Cl, | EP-A 11 18 609 |
| | | | 4-OCH(CH₃)—C(CH₃)═NOCH₂CH₃ | |

TABLE III

| | |
|---|---|
| | IV |
| 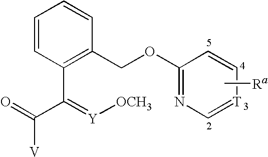 | |

| No. | V | Y | T | R^{a} | Reference |
|---|---|---|---|---|---|
| III-1 | OCH₃ | CH | N | 2-OCH₃, | WO 96/16047 |
| | | | | 4-CF₃ | |
| III-2 | OCH₃ | CH | N | 2-OCH(CH₃)₂, | WO 96/16047 |
| | | | | 4-CF₃ | |
| III-3 | OCH₃ | CH | CH | 2-CF₃ | EP-A 278 595 |
| III-4 | OCH₃ | CH | CH | 4-CF₃ | EP-A 278 595 |
| III-5 | NHCH₃ | N | CH | 2-Cl | EP-A 398 692 |
| III-6 | NHCH₃ | N | CH | 2-CF₃ | EP-A 398 692 |
| III-7 | NHCH₃ | N | CH | 2-CF₃, | EP-A 398 692 |
| | | | | 4-Cl | |
| III-8 | NHCH₃ | N | CH | 2-Cl, | EP-A 398 692 |
| | | | | 4-CF₃ | |

TABLE IV

| | |
|---|---|
| | V |
| 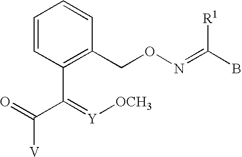 | |

| No. | V | Y | R¹ | B | Reference |
|---|---|---|---|---|---|
| IV-1 | OCH₃ | CH | CH₃ | (3-CF₃)C₆H₄ | EP-A 370 629 |
| IV-2 | OCH₃ | CH | CH₃ | (3,5-Cl₂)C₆H₃ | EP-A 370 629 |
| IV-3 | NHCH₃ | N | CH₃ | (3-CF₃)C₆H₄ | WO 92/13830 |
| IV-4 | NHCH₃ | N | CH₃ | (3-OCF₃)C₆H₄ | WO 92/13830 |
| IV-5 | OCH₃ | N | CH₃ | (3-OCF₃)C₆H₄ | EP-A 460 575 |
| IV-6 | OCH₃ | N | CH₃ | (3-CF₃)C₆H₄ | EP-A 460 575 |
| IV-7 | OCH₃ | N | CH₃ | (3,4-Cl₂)C₆H₃ | EP-A 460 575 |
| IV-8 | OCH₃ | N | CH₃ | (3,5-Cl₂)C₆H₃ | EP-A 463 488 |
| IV-9 | OCH₃ | CH | CH₃ | CH═CH-(4-Cl)C₆H₄ | EP-A 936 213 |

TABLE V

| | |
|---|---|
| | VI |
| 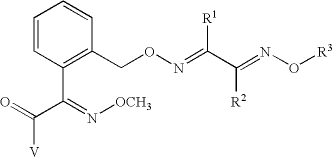 | |

| No. | V | R¹ | R² | R³ | Reference |
|---|---|---|---|---|---|
| V-1 | OCH₃ | CH₃ | CH₃ | CH₃ | WO 95/18789 |
| V-2 | OCH₃ | CH₃ | CH(CH₃)₂ | CH₃ | WO 95/18789 |
| V-3 | OCH₃ | CH₃ | CH₂CH₃ | CH₃ | WO 95/18789 |
| V-4 | NHCH₃ | CH₃ | CH₃ | CH₃ | WO 95/18789 |
| V-5 | NHCH₃ | CH₃ | 4-F—C₆H₄ | CH₃ | WO 95/18789 |
| V-6 | NHCH₃ | CH₃ | 4-Cl—C₆H₄ | CH₃ | WO 95/18789 |
| V-7 | NHCH₃ | CH₃ | 2,4-C₆H₃ | CH₃ | WO 95/18789 |
| V-8 | NHCH₃ | Cl | 4-F—C₆H₄ | CH₃ | WO 98/38857 |
| V-9 | NHCH₃ | Cl | 4-Cl—C₆H₄ | CH₂CH₃ | WO 98/38857 |
| V-10 | NHCH₃ | CH₃ | CH₂C(═CH₂)CH₃ | CH₃ | WO 97/05103 |
| V-11 | NHCH₃ | CH₃ | CH═C(CH₃)₂ | CH₃ | WO 97/05103 |
| V-12 | NHCH₃ | CH₃ | CH═C(CH₃)₂ | CH₂CH₃ | WO 97/05103 |
| V-13 | NHCH₃ | CH₃ | CH═C(CH₃)CH₂CH₃ | CH₃ | WO 97/05103 |
| V-14 | NHCH₃ | CH₃ | O—CH(CH₃)₂ | CH₃ | WO 97/06133 |
| V-15 | NHCH₃ | CH₃ | O—CH₂CH(CH₃)₂ | CH₃ | WO 97/06133 |
| V-16 | NHCH₃ | CH₃ | C(CH₃)═NOCH₃ | CH₃ | WO 97/15552 |

TABLE VI

| | |
|---|---|
| | VII |
| 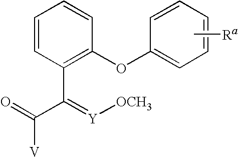 | |

| No. | V | Y | R^{a} | Reference |
|---|---|---|---|---|
| VI-1 | NHCH₃ | N | H | EP-A 398 692 |
| VI-2 | NHCH₃ | N | 3-CH₃ | EP-A 398 692 |
| VI-3 | NHCH₃ | N | 2-NO₂ | EP-A 398 692 |
| VI-4 | NHCH₃ | N | 4-NO₂ | EP-A 398 692 |
| VI-5 | NHCH₃ | N | 4-Cl | EP-A 398 692 |
| VI-6 | NHCH₃ | N | 4-Br | EP-A 398 692 |

TABLE VII

| | |
|---|---|
| | VIII |
| 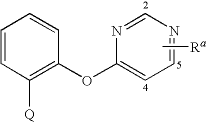 | |

| No. | Q | R^{a} | Reference |
|---|---|---|---|
| VII-1 | C(═CH—OCH₃)COOCH₃ | 5-O-(2-CN—C₆H₄) | EP-A 382 375 |
| VII-2 | C(═CH—OCH₃)COOCH₃ | 5-O-(2-Cl—C₆H₄) | EP-A 382 375 |
| VII-3 | C(═CH—OCH₃)COOCH₃ | 5-O-(2-CH₃—C₆H₄) | EP-A 382 375 |
| VII-4 | C(═N—OCH₃)CONHCH₃ | 5-O-(2-Cl—C₆H₄) | GB-A 2253624 |
| VII-5 | C(═N—OCH₃)CONHCH₃ | 5-O-(2,4-Cl₂—C₆H₃) | GB-A 2253624 |
| VII-6 | C(═N—OCH₃)CONHCH₃₃ | 5-O-(2-CH₃—C₆H₄) | GB-A 2253624 |
| VII-7 | C(═N—OCH₃)CONHCH₃ | 5-O-(2-CH₃, 3-Cl—C₆H₃) | GB-A 2253624 |
| VII-8 | C(═N—OCH₃)CONHCH₃ | 4-F, | WO 98/21189 |
| | | 5-O-(2-CH₃—C₆H₄) | |
| VII-9 | C(═N—OCH₃)CONHCH₃ | 4-F, | WO 98/21189 |
| | | 5-O-(2-Cl—C₆H₄) | |
| VII-10 | C(═N—OCH₃)CONHCH₃ | 4-F, | WO 98/21189 |
| | | 5-O-(2-CH₃, 3-Cl—C₆H₃) | |
| VII-11 | Q1 | 4-F, | WO 97/27189 |
| | | 5-O-(2-Cl—C₆H₄) | |
| VII-12 | Q1 | 4-F, | WO 97/27189 |
| | | 5-O-(2-CH₃, | |
| | | 3-Cl—C₆H₃) | |
| VII-13 | Q1 | 4-F, | WO 97/27189 |
| | | 5-O-(2,4-Cl₂—C₆H₃) | |

Particularly preferred are combinations of one of the following components 1: Compound I-5 (pyraclostrobin), II-1 (kresoxim-methyl), II-3 (dimoxystrobin), II-11 (ZJ 0712), III-3 (picoxystrobin), IV-6 (trifloxystrobin), IV-9 (enestroburin), V-16 (orysastrobin), VI-1 (metominostrobin), VII-1 (azoxystrobin), and VII-11 (fluoxastrobin) with one of the compounds selected from the groups A), B), C), and D).

In one embodiment of the invention the combination of pyraclostrobin and one of the compounds selected from the groups A), B), C), and D) is used.

In another embodiment of the invention the combination of kresoxim-methyl and one of the compounds selected from the groups A), B), C), and D) is used.

In another embodiment of the invention the combination of dimoxystrobin and one of the compounds selected from the groups A), B), C), and D) is used.

In another embodiment of the invention the combination of ZJ 0712 and one of the compounds selected from the groups A), B), C), and D) is used.

In another embodiment of the invention the combination of picoxystrobin and one of the compounds selected from the groups A), B), C), and D) is used.

In another embodiment of the invention the combination of trifloxystrobin and one of the compounds selected from the groups A), B), C), and D) is used.

In another embodiment of the invention the combination of enestroburin and one of the compounds selected from the groups A), B), C), and D) is used.

In another embodiment of the invention the combination of orysastrobin and one of the compounds selected from the groups A), B), C), and D) is used.

In another embodiment of the invention the combination of metominostrobin and one of the compounds selected from the groups A), B), C), and D) is used.

In another embodiment of the invention the combination of azoxystrobin and one of the compounds selected from the groups A), B), C), and D) is used.

In another embodiment of the invention the combination of fluoxastrobin and one of the compounds selected from the groups A), B), C), and D) is used.

In another embodiment of the invention the combination of a compound of formula I with antibiotics, especially cycloheximid, griseofulvin, kasugamycin, natamycin, polyoxin or streptomycin is used.

In another embodiment of the invention the combination of a compound of formula I with famoxadone or cymoxanil is used.

In a further embodiment of the invention the combination of a compound of formula I with copper fungicides is used.

The combination of active ingredients 1) and 2) increase the tolerance of plants to bacterioses. They are especially important for controlling bacteria on a variety of crop plants such as vegetables, top fruit species and tobacco, and all the seeds of these plants.

Specifically, they are suitable for controlling the following plant diseases: Pseudomonas species on tobacco, potatoes, tomatoes and pulses, and, in particular, Erwinia species on fruit, vegetables and potatoes.

The active ingredients 1) and 2) are applied by treating the plants, seeds or the soil to be protected from bacterial infection with an effective amount of the active ingredients. Application takes place before the bacteria infect the plants or seeds.

Preferably the active ingredients are applied shortly after germination of the plants, especially within the first four weeks after germination, long before first protective application against fungi usually is made. Best efficacy is observed, when the application is repeatedly carried out, preferably any repetition is made every 10 to 20 days.

In a preferred embodiment of the method, the plant is treated before infection takes place, preferably several weeks to one week before the expected bacteria attack. During such timeframe one to 10 applications are carried out. After repeated application a markedly reduced susceptibility of the plant to bacterioses is observed.

The method according to the invention is preferably carried out as foliar application when applied to fruit and vegetables, such as potatoes, tomatoes, cucurbits, preferably cucumbers, melons, watermelons, garlic, onions, and lettuce. Preferably more than two applications, and up to 10 applications during a season are carried out.

The method according to the invention is preferably carried out as foliar application when applied to fruits, such as apples, stone fruits, and citrus. Preferably more than two applications, and up to 5 applications during a season are carried out.

The method of the invention can also be applied to field crops, such as soybeans, corn, cotton, tobacco, common beans, wheat, barley, peas, and others. In relation to these crops the method is preferably applied by treating the seeds or the plants. The plants are preferably treated with one to three applications.

The component 1) and the component 2) can be applied simultaneously, that is jointly or separately, or in succession, the sequence, in the case of separate application, generally not having any effect on the result of the control measures.

In one embodiment of the mixtures according to the invention, a further active compound 3) or two active compounds 3) and 4) are added to the components 1) and 2). the active compounds useful as components 3) and 4) are preferably those which are mentioned as component 2) in the outset, or a compound selected from the group
amine derivatives such as aldimorph, dodine, dodemorph, fenpropimorph, fenpropidin, guazatine, iminoctadine, spiroxamin, tridemorph
anilinopyrimidines such as pyrimethanil, mepanipyrim or cyrodinyl,
azoles such as bitertanol, bromoconazole, cyproconazole, difenoconazole, dinitroconazole, enilconazole, epoxiconazole, fenbuconazole, fluquiconazole, flusilazole, flutriafol, hexaconazole, imazalil, metconazole, myclobutanil, penconazole, propiconazole, prochloraz, prothioconazole, tebuconazole, triadimefon, triadimenol, triflumizol, triticonazole,
dicarboximides such as iprodion, myclozolin, procymidon, vinclozolin,
dithiocarbamates such as ferbam, nabam, maneb, mancozeb, metam, metiram, propineb, polycarbamate, thiram, ziram, zineb,
heterocyclic compounds such as anilazine, benomyl, boscalid, carbendazim, carboxin, oxycarboxin, cyazofamid, dazomet, dithianon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine, 5-Chloro-7-(4-methylpiperidin-1-yl)-6-(2,4,6-trifluoro-phenyl)-[1,2,4]triazolo[1,5-a]pyrimidine,
nitrophenyl derivatives such as binapacryl, dinocap, dinobuton, nitrophthalisopropyl
phenylpyrroles such as fenpiclonil or fludioxonil,
sulfur,
other fungicides such as acibenzolar-S-methyl, benthiavalicarb, carpropamid, chlorothalonil, cyflufenamid, dazomet, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, mandipropamide, metrafenon, pencycuron, phosphorous acid and its salts, propamocarb, phthalide, toloclofos-methyl, quintozene, zoxamide,
sulfenic acid derivatives such as captafol, captan, dichlofluanid, folpet, tolylfluanid,
cinnemamides and analogs such as dimethomorph, flumetover or flumorph.

Preference is given to combinations of the components 1) and 2) and a component 3). Particular preference is given to combinations of the components 1) and 2).

The component 1) and the component 2) are usually applied in a weight ratio of from 100:1 to 1:100, preferably from 20:1 to 1:20, in particular from 10:1 to 1:10.

The components 3) and, if appropriate, 4) are, if desired, added in a ratio of 20:1 to 1:20 to the component 1).

Preferred components 3) are fungicides from the dithiocarbamate class.

Depending on the type of compound and the desired effect, the application rates of the mixtures according to the invention are from 5 g/ha to 1000 g/ha, preferably from 50 to 900 g/ha, in particular from 50 to 750 g/ha.

Correspondingly, the application rates for the component 1) are generally from 1 to 1000 g/ha, preferably from 10 to 900 g/ha, in particular from 20 to 750 g/ha.

Correspondingly, the application rates for the component 2) are generally from 1 to 1000 g/ha, preferably from 10 to 500 g/ha, in particular from 40 to 350 g/ha.

In the treatment of seed, application rates of mixture are generally from 1 to 1000 g/100 kg of seed, preferably from 1 to 200 g/100 kg, in particular from 5 to 100 g/100 kg.

The mixtures according to the invention, or the components 1) and 2), can be converted into the customary formulations, for example solutions, emulsions, suspensions, dusts, powders, pastes and granules. The use form depends on the particular intended purpose; in each case, it should ensure a fine and even distribution of the compound according to the invention.

Best results are obtained when a formulation is used which supports the penetration of the active compounds into the plants, and the distribution within the entire plant in the sap. Such especially suitable formulations are, e.g. EC, DC, SE.

The compounds 1) and 2) can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound may also be used successfully in the ultra-low-volume process (ULV), it being possible to apply formulations comprising over 95% by weight of active compound, or even to apply the active compound without additives.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents. The use of formulations of copper salts which contain basic amino acids, lysin, polylysin, or polylysin derivatives is not subject of the current invention.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example Dichlorophen and enzylalkoholhemiformal.

Seed Treatment formulations may additionally comprise binders and optionally colorants.

Binders can be added to improve the adhesion of the active materials on the seeds after treatment. Suitable binders are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The following are examples of formulations: 1. Products for dilution with water for foliar applications. For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

### B) Dispersible Concentrates (DC)

20 parts by weight of the active compound(s) are dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

25 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

2. Products to be applied undiluted for foliar applications. For seed treatment purposes, such products may be applied to the seed diluted

### I) Dustable Powders (DP, DS)

5 parts by weight of the active compound(s) are ground finely and mixed intimately with 95 parts by weight of finely divided kaolin. This gives a dustable product having 5% (w/w) of active compound(s)

### J) Granules (GR, FG, GG, MG)

0.5 part by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

### K) ULV Solutions (UL)

10 parts by weight of the active compound(s) are dissolved in 90 parts by weight of an organic solvent, for example xylene. This gives a product having 10% (w/w) of active compound(s), which is applied undiluted for foliar use.

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulation can be applied to the seed diluted or undiluted. Application to the seeds is carried out before sowing, either directly on the seeds.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Oils of various types, wetters, adjuvants, herbicides, fungicides, other pesticides, or bactericides may be added to the active compounds, even, if appropriate, not until immediately prior to use (tank mix). These agents are typically admixed with the compositions according to the invention in a weight ratio of from 1:10 to 10:1.

The note mentioning the effect of the active ingredients 1) and 2) in inducing tolerance to bacteria may be present as a label on the packaging or in product data sheets. The note may also be present in the case of preparations, which can be used in combination with the active ingredients 1) and 2).

The induction of tolerance may also constitute an indication which may be the subject of official approval of combinations of active ingredients 1) and 2).

The action of the mixtures according to the invention was demonstrated by the following experiments:

### USE EXAMPLES FOR INDUCTION OF TOLERANCE TO BACTERIOSES

The active compounds used were applied as commercially available formulations. Pyraclostrobin was used as Comet®, an EC of BASF Aktiengesellschaft, Pyraclostrobin+Metiram was used as Cabrio Top®, a WG of BASF Aktiengesellschaft, copper oxychloride was used as Cobox DF™, a 87% WG of Quimetal Industrial S.A. (Chile)

### Use Example 1

### Preventive Action on Potatoes Against Erwinia carotovora

The trial was conducted under field conditions. Potato plants of variety Bintje were planted as tubers and grown under standard conditions with adequate supply of water and nutrients. After 37 days a first application of active compounds was made, which was repeated four times every 5 to 7 days. No other compounds were applied for pathogen control. Infection with pathogens occurred naturally. Each treatment consisted of four replicants in a randomized block design. The disease incidences were evaluated 29 days after the first application (Erwinia carotovora). The dosage rates used and the obtained results are shown below:

The visually determined percentages of infected leaf areas were converted into efficacies in % of the untreated control:

The efficacy (E) is calculated as follows using Abbot's formula:

E=(1−α/β)·100

α corresponds to the fungal infection of the treated plants in % and
β corresponds to the fungal infection of the untreated (control) plants in %

An efficacy of 0 means that the infection level of the treated plants corresponds to that of the untreated control plants; an efficacy of 100 means that the treated plants were not infected.

The expected efficacies of mixtures of active compounds were determined using Colby's formula (Colby, S. R. “Calculating synergistic and antagonistic responses of herbicide combinations”, Weeds, 15, 20-22, 1967) and compared with the observed efficacies.

Colby's formula:

E=x+y−x·y/100
E expected efficacy, expressed in % of the untreated control, when using the mixture of the active compounds A and B at the concentrations a and b
x efficacy, expressed in % of the untreated control, when using the active compound A at the concentration a
y efficacy, expressed in % of the untreated control, when using the active compound B at the concentration b

| | | Application rate of | Efficacy in % |
|---|---|---|---|
| | | active ingredient | of the untreated |
| Example | Active compound | [g/ha] | control |
| 1 | Control (untreated) | — | (73% |
| | | | infection) |
| 2 | Pyraclostrobin | 150 | 75 |
| 3 | Pyraclostrobin + | 150 + 2600 | 89 |
| | copper oxychloride | | |

The efficacy of copper against Erwinia carotovora under the trial conditions is assumed very close to zero.

### Use Example 2

### Preventive Action on Tomatoes Against Xathomonas ssp

The trial was conducted under field conditions. Tomato plants of variety Carmen at a height of app. 10 cm were planted and grown under standard conditions with adequate supply of water and nutrients. After 18 days a first application of active compounds was made, which was repeated five times every 5 to 8 days. No other compounds were applied for pathogen control. Infection with pathogens occurred. Each treatment consisted of four replicants in a randomized block design. The disease incidences were evaluated 46 days after the first application (Xathomonas ssp.). The dosages used and the obtained results are shown below:

| | | Application rate of | Efficacy in % |
|---|---|---|---|
| | | active ingredient | of the untreated |
| Example | Active compound | [g/ha] | control |
| 4 | Control (untreated) | — | (20% |
| | | | infection) |
| 5 | Pyraclostrobin + | 175 + 1745 | 75 |
| | Metiram | | |
| 6 | Pyraclostrobin + | 175 + 1745 + 2090 | 95 |
| | Metiram + | | |
| | copper oxychloride | | |

The efficacy of copper against Xathomonas under the trial conditions is assumed very close to zero.

1. A method of inducing tolerance against bacterioses of plants which comprises treating the plants, the soil or seeds with an effective amount of a combination of
1) a strobilurin compound of the formula I 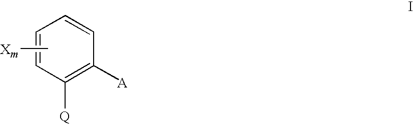
in which
X is halogen, C₁-C₄-alkyl or trifluoromethyl;
m is 0 or 1;
Q is C(═CH—CH₃)—COOCH₃, C(═CH—OCH₃)—COOCH₃, C(═N—OCH₃)—CONHCH₃, C(═N—OCH₃)—COOCH₃, N(—OCH₃)—COOCH₃, or a group Q1 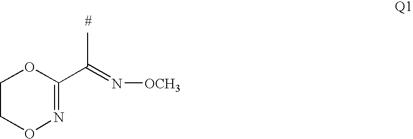
wherein # denotes the bond to the phenyl ring;
A is —O—B, —CH₂O—B, —OCH₂—B, —CH═CH—B, —C≡C—B, —CH₂O—N═C(R¹)—B, —CH₂O—N═C(R¹)—CH═CH—B, or —CH₂O—N═C(R¹)—C(R²)═N—OR³, where
B is phenyl, naphthyl, 5-membered or 6-membered hetaryl or 5-membered or 6-membered heterocyclyl, containing one to three N atoms and/or one O or S atom or one or two O and/or S atoms, the ring systems being unsubstituted or substituted by one to three radicals R^{a}:
R^{a} is cyano, nitro, amino, aminocarbonyl, aminothiocarbonyl, halogen, C₁-C₆-alkyl, C₁-C₆-haloalkyl, C₁-C₆-alkylcarbonyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkyloxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy, phenyl, phenoxy, benzyl, benzyloxy, 5- or 6-membered heterocyclyl, 5- or 6-membered hetaryl, 5- or 6-membered hetaryloxy, C(═NOR^{a})—R^{b} or OC(R^{a})₂—C(R^{b})═NOR^{b}, the cyclic radicals, in turn, being unsubstituted or substituted by one to three radicals R^{b}:
R^{b} is cyano, nitro, halogen, amino, aminocarbonyl, aminothiocarbonyl, C₁-C₆-alkyl, C₁-C₆-haloalkyl, C₁-C₆-alkylsulfonyl, C₁-C₆-alkylsulfinyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkyl-aminothiocarbonyl, C₂-C₆-alkenyl, C₂-C₆-alkenyloxy, C₃-C₆-cycloalkyl, C₃-C₆-cycloalkenyl, phenyl, phenoxy, phenylthio, benzyl, benzyloxy, 5- or 6-membered heterocyclyl, 5- or 6-membered hetaryl, 5- or 6-membered hetaryloxy or C(═NOR^{A})—R^{B};
R^{A}, R^{B} are hydrogen or C₁-C₆-alkyl;
R¹ is hydrogen, cyano, C₁-C₄-alkyl, C₁-C₄-haloalkyl, C₃-C₆-cycloalkyl, C₁-C₄-alkoxy;
R² is phenyl, phenylcarbonyl, phenylsulfonyl, 5- or 6-membered hetaryl, 5- or 6-membered hetarylcarbonyl or 5- or 6-membered hetarylsulfonyl, the ring systems being unsubstituted or substituted by one to three radicals R^{a},
C₁-C₁₀-alkyl, C₃-C₆-cycloalkyl, C₂-C₁₀-alkenyl, C₂-C₁₀-alkynyl, C₁-C₁₀-alkylcarbonyl, C₂-C₁₀-alkenylcarbonyl, C₃-C₁₀-alkynylcarbonyl, C₁-C₁₀-alkylsulfonyl, or C(═NOR^{a})—R^{b}, the hydrocarbon radicals of these groups being unsubstituted or substituted by one to three radicals R^{c}:
R^{c} is cyano, nitro, amino, aminocarbonyl, aminothiocarbonyl, halogen, C₁-C₆-alkyl, C₁-C₆-haloalkyl, C₁-C₆-alkylsulfonyl, C₁-C₆-alkylsulfinyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₁-C₆-alkylaminothiocarbonyl, di-C₁-C₆-alkylaminothiocarbonyl, C₂-C₆-alkenyl, C₂-C₆-alkenyloxy,
C₃-C₆-cycloalkyl, C₃-C₆-cycloalkyloxy, 5- or 6-membered heterocyclyl, 5- or 6-membered heterocyclyloxy, benzyl, benzyloxy, phenyl, phenoxy, phenylthio, 5- or 6-membered hetaryl, 5- or 6-membered hetaryloxy and hetarylthio, it being possible for the cyclic groups, in turn, to be partially or fully halogenated or to have attached to them one to three radicals R^{a}; and
R³ is hydrogen, C₁-C₆-alkyl, C₂-C₆-alkenyl, C₂-C₆-alkynyl, the hydrocarbon radicals of these groups being unsubstituted or substituted by one to three radicals R^{c};
and
2) a compound selected from the groups A) to D):
A) antibiotics: cycloheximid, griseofulvin, kasugamycin, natamycin, polyoxin and streptomycin,
B) bactericides: bronopol, cresol, dichlorophen, dipyrithione, dodicin, fenaminosulf, formaldehyde, hydrargaphen, 8-hydroxyquinoline sulfate, nitrapyrin, octhilinone, oxolinic acid, oxytetracycline, probenazole, tecloftalam, thiomersal;
C) famoxadone and cymoxanil, and
D) copper, silver, and zinc salts, such as Bordeaux mixture, copper acetate, copper oxychloride, basic copper sulfate;
in synergistically effective amounts, which active compounds 1) and 2) are taken up by the plants or seeds.

2. A method as claimed in claim 1, wherein component 1) is selected from: pyraclostrobin, kresoxim-methyl, dimoxystrobin, ZJ 0712, picoxystrobin, trifloxystrobin, enestroburin, orysastrobin, metominostrobin, azoxystrobin, and fluoxastrobin.

3. A method as claimed in claim 1, wherein component 1) is pyraclostrobin.

4. A method as claimed in claim 1, wherein component 2) is selected from cycloheximid, griseofulvin, kasugamycin, natamycin, polyoxin or streptomycin.

5. A method as claimed in claim 1, wherein component 2) is selected from famoxadone, and cymoxanil.

6. A method as claimed in claim 1, wherein component 2) is selected from copper fungicides.

7. A method as claimed in claim 5, wherein the copper fungicide is selected from Bordeaux mixture, copper acetate, copper oxychloride, and basic copper sulfate.

8. A method as claimed in claim 1, wherein components 1) and 2) are used in ratios of from 100:1 to 1:100.

9. A method as claimed in claim 1 wherein application of components 1) and 2) is carried out during the first six weeks of the growth period of the plants.

10. A method as claimed in claim 1 wherein application of components 1) and 2) is carried out within the first four weeks after germination of the plants.

11. A method as claimed in claim 1 wherein application of components 1) and 2) is carried out one to ten times before expected bacteria attack.

12. A method as claimed in claim 1 wherein components 1) and 2) are applied to potato, or tomato plants.

13. A method as claimed in claim 1 wherein components 1) and 2) are applied to seeds.

14. The use of the combinations as defined in claim 1 for inducing bacteria tolerance in plants.

15. A method as claimed in claim 2, wherein component 1) is pyraclostrobin.

16. A method as claimed in claim 2, wherein component 2) is selected from cycloheximid, griseofulvin, kasugamycin, natamycin, polyoxin or streptomycin.

17. A method as claimed in claim 2, wherein component 2) is selected from famoxadone, and cymoxanil.

18. A method as claimed in claim 2, wherein component 2) is selected from copper fungicides.

19. A method as claimed in claim 2, wherein components 1) and 2) are used in ratios of from 100:1 to 1:100.

20. A method as claimed in claim 3, wherein components 1) and 2) are used in ratios of from 100:1 to 1:100.
